(19) United States
(12) Patent Application Publication
(10) Pub. No.: US 2026/0294305 A1
(43) Pub. Date: Oct. 1, 2026

(54) METHOD FOR EX VIVO MEASURING GLOMERULAR FILTRATION RATE IN A SUBJECT

(71) Applicant: Assistance Publique - Hôpitaux de Paris, Paris (FR)

(72) Inventors: Thomas STEHLE, Créteil (FR); Frédéric PIGNEUR, Créteil (FR); Felix WEI, Créteil (FR)

(21) Appl. No.: 19/489,830

(22) PCT Filed: Jun. 14, 2024
(86) PCT No.: PCT/EP2024/066516

§ 371 Date: Dec. 3, 2025

(30) Foreign Application Priority Data
Jun. 14, 2023 (EP) 23305943.5

(51) Int. Cl. A61B 5/20; A61B 5/055; A61B 6/00

(52) U.S. Cl. CPC A61B 5/201; A61B 5/055; A61B 6/481; A61B 6/5217

(57) ABSTRACT

The present invention relates to a method for ex vivo measuring glomerular filtration rate (GFR) of a subject to whom a contrast agent has been administered, and to whom computed tomography (CT) or magnetic resonance imaging (MRI) including an excretory phase, an unenhanced phase, an arterial phase, and a nephrographic or a venous portal phase has been performed, said method comprising the step of calculating the clearance of the contrast agent from CT or MRI data using manual, automated or semi-automated segmentation software, thereby obtaining a measured GFR.

### CROSS-REFERENCE TO RELATED APPLICATIONS

The present application is a filing under 35 U.S.C. 371 as the National Stage of International Application No. PCT/EP2024/066516, filed Jun. 14, 2024, entitled “METHOD FOR EX VIVO MEASURING GLOMERULAR FILTRATION RATE IN A SUBJECT,” which claims priority to European Application No. 23305943.5 filed with the European Patent Office on Jun. 14, 2023, both of which are incorporated herein by reference in their entirety for all purposes.

### TECHNICAL FIELD

The present invention refers to a method for ex vivo measuring glomerular filtration rate (GFR) in a subject.

Therefore, the present invention has utility in medical field, in particular in the fields of diagnosis and patient monitoring.

In the description below, the references into brackets ([ ]) refer to the listing of references situated at the end of the text.

### BACKGROUND OF THE INVENTION

Glomerular filtration rate (GFR) is the main variable used to assess kidney function. It is used in particular for the dosage adjustment of drugs excreted by the kidneys, or to make various clinical decisions. In clinical practice, GFR is estimated from equations based on serum or plasma concentrations of endogenous markers, such as creatinine or cystatin C. One major limitation of estimated GFR (eGFR) is its inaccuracy: none of the equations based on creatinine and/or cystatin C have an accuracy within 30% significantly above 90% over the entire age spectrum. This means that in at least one in ten patients, eGFR over- or underestimates measured GFR (mGFR) by more than 30% or less than-30%. Risk factors for inaccuracy of creatinine-based eGFR are conditions that impact non-GFR determinants of serum creatinine concentration, i.e., atypical muscle mass, a very high-protein or conversely vegetarian diet, or the use of medications that block tubular creatinine secretion. In cases when knowing the true GFR value is needed for clinical decision making, GFR can be measured by assessing the clearance of an exogenous tracer (Ebert N et al. [1])). These procedures are cumbersome, human resource and time consuming (at least 4 to 5 hours, and up to 24 hours in some cases), and thus costly.

The development of simple methods for measuring GFR, which would be rapid and reproducible, is an unmet need to date. Even if old and recent studies have assessed the practicability of measuring GFR from CT scans using various specifically dedicated acquisition protocol (Yuan X et al. ([2]), You S et al. ([3]), Hackstein N et al. ([4]), Hackstein N et al. ([5]), Jeong S, Park S B et al. ([6])), they are not necessarily those used in routine care, which may explain why they have not been widely expanded. Historically, two-compartment mathematical models have first been used. Most of them were based on the Patlak plot model, which was originally developed to measure constant transfers across the blood-brain barrier (Patlak C S et al. ([7])). In this model, which considers only the vascular and tubular compartments, it was assumed that the tracer diffuses irreversibly from the vascular compartment to the tubular compartment with a transfer coefficient which corresponds to the GFR. The interstitial sector, quantitatively variable from one subject to another, was thus not taken into account. The tracer exit towards the excretory tracts was not considered either. This model, whether original or modified, did not perform very well in measuring GFR (Hackstein N et al. ([4]), Hackstein N et al. ([5])). More recently, 2 groups have proposed methods for measuring GFR (iopromide clearance) based on both CT scan and biology (hematocrit) (Yuan X et al. ([2]), You S et al. ([3])). Yuan et al showed a very good agreement with plasma clearance of ^{99m}Tc-DTPA in 42 patients, using perfusion CT scans with multiphase dynamic acquisitions (Yuan X et al. ([2])), an acquisition method that is not used in clinical routine. You et al protocol also had very strictly defined acquisition parameters, with unenhanced, nephrographic (100 sec post bolus injection, precisely) and excretory phases (600 sec, precisely) (You S et al. ([3])). This protocol appears to have poorer agreement versus a GFR measured by the Gates method (You S et al. ([3])), which itself is not a reference method for GFR measurement, being potentially no more accurate than a creatinine-based eGFR (Yuan X et al. ([2]), Aydin F et al. ([8]), Itoh K. ([9]), Kumar M et al. ([10])). These 2 methods for measuring GFR share the disadvantage of not being retrospectively usable from CT urography performed in routine care. In addition, in both cases the value of the hematocrit was included in the calculation of the GFR, which also makes them somewhat impractical.

Thus, a need exists of alternative methods for measuring GFR. The present invention fulfills these and other needs.

### DESCRIPTION OF THE INVENTION

The Applicant has found surprisingly that glomerular filtration rate (GFR) can be measured by assessing a contrast agent clearance from opportunistic imaging, such as computed tomography (CT) or magnetic resonance imaging (MRI), in a subject.

Surprisingly, segmentation of various areas of interest at different relevant time points of opportunistic imaging allows calculation of a contrast agent clearance, i.e. glomerular filtration rate in a subject.

The inventors showed that GFR measured by opportunistic imaging was unbiased and had excellent agreement with GFR measured by laboratory iohexol clearance (gold standard).

After intensive research, the inventors succeeded in developing a method in which measured GFR was reliable despite the wide variability imaging procedures, demonstrating its utility, especially in an opportunistic value-added imaging approach.

Accordingly, in a first aspect, the present invention provides a method for ex vivo measuring GFR in a subject, comprising the steps of:
1) administering a contrast agent to the subject,
2) performing an imaging, such as computed tomography (CT) or magnetic resonance imaging (MRI), including an excretory phase, an unenhanced phase, an arterial phase, and a nephrographic or a venous portal phase to the subject,
3) Calculating the clearance of the contrast agent from CT or MRI data obtained in step 2) using manual, automated or semi-automated segmentation software, thereby obtaining a measured GFR.

In other words, the present invention provides a method for ex vivo measuring GFR of a subject to whom a contrast agent has been administered and to whom CT or MRI, including, an unenhanced phase, an arterial phase, a nephrographic or a venous portal phase, and an excretory phase has been performed, said method comprising the step of calculating the clearance of the contrast agent from CT or MRI data using manual, automated or semi-automated segmentation software, thereby obtaining a measured GFR.

The contrast agent, also called herein indifferently “contrast media”, may be an ideal exogenous marker for measuring GFR, that would diffuse freely into the extracellular volume, not be metabolized nor bound to plasma proteins, be eliminated exclusively by the kidney, as it would be freely filtered, and not secreted or reabsorbed in the tubule. It may be any a substance allowing to increase the contrast of structures or fluids within the body in medical imaging. The contrast agent may be for example iodinated or non-iodinated agents. They may be for example iodine, or gadolinium-based contrast agent. Iodine-based contrast agents may be for example chosen among iomeprol, iohexol, iopamidol, ioxilan, iopromide, iodixanol, iobitridol, ioversol, diatrizoate, metrizoate, iothalamate, ioxaglate. Gadolinium-based contrast agents may be for example chosen among gadopentetate dimeglumine (gadolinium diethylene triamine pentaacetic acid (Gd-DTPA), gadodiamide (gadolinium diethylene triamine penta-acetic acid bis-methylamide (GD-DTPA-BMA), Gadoteridol (Gadolinium-1,4,7-tris (carboxymethyl)-10-(2′ hydroxypropyl)-1, 4, 7-10-tetraazacyclododecane (Gd-HPD03A]), gadoterate meglumine (gadolinium-tetraazacyclododecane tetra acetic acid (Gd-DOTA), Gadoteric acid such as Dotarem® or clariscan, gadobenic acid and their salts such as gadobenate dimeglumine, gadoxetic acid and gadobutrol.

Advantageously, the contrast agent used may be iomeprol. Iomeprol is a nonionic, hydrosoluble, iodinated contrast agent with low viscosity and low osmolality. Its molecular weight of 777.09 Dalton is slightly lower than that of iohexol (821.1 Da) or iothalamate (809.1 Da). Furthermore, iomeprol does not bind measurably to plasma proteins. These chemical properties make iomeprol potentially an ideal exogenous marker for measuring GFR, i.e. it would diffuse freely into the extracellular volume, not be metabolized nor bound to plasma proteins, be eliminated exclusively by the kidney, as it would be freely filtered, and not secreted or reabsorbed in the tubule. Pharmacokinetic studies confirmed that after a bolus injection of iomeprol, its plasma concentration declined biexponentially (first slope corresponding to its diffusion in the extracellular volume and its renal clearance, and second slope corresponding only to its renal clearance), with a negligible extra-renal clearance, and a very good correlation between its clearance and that of inulin.

Administration of the contrast agent to the subject may be performed by any classical route, depending the kind of the agent. It may be for example intravenous. Preferably, the contrast agent is administered to the subject prior to implementation of the method of the invention, i.e., the step of administering the contrast agent is not part of the method of the invention.

The concentration in the blood, urine and/or renal parenchyma of administered contrast agent may be determined by any method known in the state of the art. It may be Hounsfield unit count, or a different method such as iodine quantification on spectral CT detector, photon-counting CT, or MRI signal intensity. It has to be noted that spectral imaging enables direct measurement of iodine grammage without the need for spontaneous contrast acquisition. So, in case of spectral imaging and photo-counting CT imaging, the unenhanced CT phase is not required to calculate the GFR, because all the measurements made correspond to the iodine grammage, which is equal to zero on the unenhanced phase. Therefore, in case of spectral and photo-counting CT, the unenhanced CT phase contributes nothing to the results, and is therefore optional. In other words, in case of spectral imaging, only an excretory phase, an arterial phase, and a nephrographic or a venous portal phase may be performed.

Advantageously, the method of the invention does not take into account the quantity of contrast agent administered, and would be applicable with reduced doses of contrast agent, for example less than 1 ml/Kg for iodinated contrast agents.

The subject may be a healthy subject or a subject with a kidney disease, for example a chronic kidney disease.

“Imaging” refers herein to any medical imaging technique and process of creating data, especially images, of the interior of a body. It may be computed tomography (CT) or magnetic resonance imaging (MRI). CT scan may be for example polychromatic computed tomography, spectral computed tomography, photon counting computed tomography, sequential CT, spiral CT, Electron beam tomography, Dual Energy CT, CT perfusion imaging or PET CT. MRI scan may be a contrast MRI, regardless of the strength of the magnetic field. Advantageously, it may be an opportunistic imaging, such as an extraction of an imaging biomarker or feature on an imaging examination performed for a clinical target different than measuring GFR. Alternatively, it may be a CT or MRI examination performed for the sole purpose of measuring GFR. Preferably, the imaging is performed to the subject prior to implementation of the method of the invention, i.e., the step of imaging is not part of the method of the invention.

“Excretory phase” refers herein to a post-contrast agent administration time range in which there is an optimal enhancement of the renal collecting systems and the bladder, which is at least about 7 minutes after injection of the contrast agent, and for example at about 10 minutes after injection of the contrast agent. Advantageously, this period allows the contrast agent to reach the bladder. In other words, it may be the period of urinary excretion of the contrast agent after glomerular filtration.

In an embodiment, the imaging may be limited to a single slice at the usual times of the arterial and the nephrographic or venous portal phases, in order to limit X-ray dose in case of CT imaging performed for the sole purpose of measuring GFR. The arterial phase may also be limited to a single slice of the aorta in healthy individuals. For patients with kidney disease, the arterial phase may be acquired preferably over the entire height of the kidneys, in order to enable the kidney cortex to be studied, and is thus not limited to a single slice. The excretory phase may include kidney parenchyma, upper kidney excretory system, and bladder.

The unenhanced phase may include the kidney parenchyma and optionally may include the bladder, partially or in its entirety, especially in case iodine concentration cannot be measured directly on post-IV acquisitions. Alternatively, when iodine concentration can be measured directly on post-IV acquisitions, particularly in spectral imaging, this acquisition is optional.

The arterial and/or nephrographic (or venous portal) phases may include at least one aortic slice. The bladder may be included partially or in its entirety in the arterial or in the nephrographic or venous portal phase if not included in the unenhanced phase. In other words, nephrographic or portal venous phases may include at least one aortic single slice, together with the bladder if not included in the unenhanced phase.

“Segmentation” refers herein to any process dividing an image into regions with similar properties, such as gray level, color, texture, brightness, and contrast, in order to subdivide the objects in an image. Automated or semi-automated segmentation software may be any software of the state of the art, such as ADW Server GE HealthCare, syngo.via Siemens, Carestream, Osirix, 3D Slicer, Total Segmentator, MONAI Auto3DSeg, this list not being limitative. For example, step 3) may be performed automatically using software developed from algorithms developed by artificial intelligence, i.e. Deep Learning, or other methods, notably to determine both the urinary excretion and the mid-period serum concentration of the contrast agent.

Step 3), i.e. step of calculating, may be performed in whole or in part by a calculator. The calculator may be any calculator that calculates the clearance of contrast agent using the method of invention. The calculator may be included or not in the automated segmentation software.

Advantageously, contrast agent clearance may be calculated by dividing the urinary excretion rate of contrast agent between arterial and excretory phases by calculated serum concentration of the contrast agent at mid period. Advantageously, this calculation method may have at least one of the following advantages:
the possibility of using CT-urography done in the context of care as part of an opportunistic imaging approach, especially compared with CT-based pharmacokinetic models such as Patlak's ([7]), which require dynamic acquisitions that are not those made for care,
better accuracy than CT-based pharmacokinetic models,
no need for additional blood tests, whereas some methods of the prior art require measuring hematocrit,
shorter examination, of about 7 to 10 min vs. at least 4 h30, sometimes 24 h for laboratory methods,
no need for blood or urine assays, or specialized pharmacology or nuclear medicine laboratories, which are required for laboratory methods.
lower cost than laboratory methods. For example, urinary clearance of an exogenous tracer requires day hospitalization, which in France costs over 1,000 euros, vs. around 150 euros for a CT scan,
less consumption of paramedical time than laboratory methods.

Therefore, as described above, the present invention provides a method for ex vivo measuring GFR of a subject to whom a contrast agent has been administered and to whom CT or MRI, including preferably, an unenhanced phase, an arterial phase, a nephrographic or a venous portal phase, and an excretory phase has been performed, said method comprising the step of calculating the clearance of the contrast agent from CT or MRI data using manual, automated or semi-automated segmentation software, thereby obtaining a measured GFR, wherein contrast agent clearance may be calculated by dividing the urinary excretion rate of contrast agent between arterial and excretory phases by calculated serum concentration of the contrast agent at mid period.

For example, the urinary excretion rate of contrast agent between arterial and excretory phases may be the sum of urinary excretion of the contrast agent into the bladder (Uexcr-Bladder), urinary excretion of the contrast agent into the upper excretory tract (Uexcr-tract), and urinary excretion of the contrast agent into the kidney tubules (Uexcr-tubules). The mid period serum contrast agent concentration may be deduced from a linear or polynomial regression after transformation of each intermediate value of serum concentration equivalents into a natural logarithm.

In an embodiment, the urinary excretion of the contrast agent into the kidney tubules (Uexcr-tubules) is the volume of both kidneys multiplied by mean attenuation of kidneys at the excretory phase (HU-Kidney-excr) minus mean attenuation of kidneys at the unenhanced phase (HU-Kidney-unenhanced), then divided by time period. Advantageously, this embodiment may be performed for subjects having normal GFR, i.e. healthy subjects or subjects having a creatinine or cystatin C-based estimated GFR at least equal to 60 ml/min/1.73 m².

In another embodiment, the urinary excretion of the contrast agent into the kidney tubules (Uexcr-tubules) is the sum of urinary excretion of the contrast agent into the kidney medulla and urinary excretion of the contrast agent into the kidney cortex. Advantageously, this embodiment may be performed out either for subjects having low GFR, i.e. ill subjects or subjects having a creatinine or cystatin C-based estimated GFR inferior to 60 ml/min/1.73 m², or for healthy subjects, because this calculation method leads for them to results that are very similar to the first aforementioned method. For example, in case of the CT:
the urinary excretion of the contrast media into the kidney medulla may be the volume of kidney medulla multiplied by mean CT-attenuation of kidneys at the excretory phase (HU-Kidney-excr) (which is the same as that of the medulla, the kidney parenchyma being homogeneous in this CT phase) minus mean attenuation of kidneys at the unenhanced phase (HU-Kidney-unenhanced) (the kidney parenchyma is also homogeneous in this CT phase), then divided by time period, and
the urinary excretion of the contrast agent into the kidney cortex may be the volume of kidney cortex multiplied by mean CT-attenuation of kidney at the excretory phase (HU-Kidney-excr) (which is the same as that of the cortex, the kidney parenchyma being homogeneous in this CT phase) minus CT-attenuation of the kidneys in the unenhanced phase, and minus cortical CT-attenuation from the vascular compartment in the excretory phase (corresponding to the presence of contrast media in the vascular compartment of the cortex in this excretory phase), then divided by time between arterial and excretory phases. The cortical CT-attenuation attributable to the vascular compartment in the excretory phase may be calculated as follows: First, determination of the ratio between the enhancement of the cortex and that of the aorta during the arterial phase, then this ratio is multiplied by the aorta contrast uptake in the excretory phase. The ratio between the enhancement of the cortex and that of the aorta during the arterial phase is CT-attenuation of the kidney cortex at the arterial phase minus CT-attenuation of the kidney at the unenhanced phase, divided by CT-attenuation of the aorta at the arterial phase minus CT attenuation of the aorta at the unenhanced phase. The aorta contrast uptake in the excretory phase is CT-attenuation of the aorta in the excretory phase minus CT-attenuation of the aorta in the unenhanced phase.

To measure the urinary excretion of contrast media in the bladder, the measurement of bladder volume and its mean attenuation at the excretory phase can involve either the total volume of the bladder, or just the part of the bladder in the declive zone that contains the contrast media.

Advantageously, specific areas of CT scan or MRI scan may be segmented at different times in order to determine urinary excretion rate of the contrast agent.

Advantageously; a volume of aorta or a single slice is used to measure CT density or MRI signal of the aorta.

This invention is further illustrated by the following examples with regard to the annexed drawings that should not be construed as limiting.

### BRIEF DESCRIPTION OF THE FIGURES

FIG. 1: represents flowchart of study population (living kidney donors (i.e. healthy individuals).

FIGS. 2A-2D: represent agreement analysis of CT-mGFR versus mGFR, and also between eGFR and mGFR. CT-mGFR is determined in healthy individuals, by calculating Uexcr-tubules without taking into account the contrast product present in the vascular compartment of the cortex. Bland Altman plots comparing CT-mGFR and mGFR (FIG. 2A), and CKD-EPI2021 and mGFR (FIG. 2B). X-axis is the mean of the results obtained with the two GFR assessment methods. Y-axis is the relative difference between the two GFR assessment methods. The solid lines are the bias (the mean relative difference) and the dashed lines are the lower and upper limits of the interval of agreement (−1.96 SD and +1.96 SD). Relationship, as assessed by Passing Bablok regression, between CT-mGFR and mGFR (FIG. 2C), and between CKD-EPI2021 and mGFR (FIG. 2D). The equations for the regression lines are indicated in the figures graphs. Dashed lines are identity lines and thick lines are the regression lines.

FIG. 3: represents intraobserver agreement assessment with Bland Altman plots for CT-mGFR determined in healthy individuals, by calculating Uexcr-tubules without taking into account the contrast product present in the vascular compartment of the cortex. X-axis is the mean of the results obtained with the two successive GFR measurements. Y-axis is the relative difference between the two GFR measurements. The solid lines are the bias (the mean relative difference) and the dashed lines are the lower and upper limits of the interval of agreement (−1.96 SD and +1.96 SD).

FIG. 4 represents the flowchart of study inclusion criteria in chronic kidney disease population.

FIGS. 5A-5E represent the Parameters derived from CT urography used to calculate the contrast media in the kidney tubules, either by considering the kidney parenchyma as a single entity, or by calculating the contrast media in the cortical tubules and the contrast media in the medullary tubules separately. CT attenuation and/or volume measurements of kidney parenchyma and/or aorta, required to calculate the urinary excretion of the contrast agent in the kidney tubules, are provided in FIGS. 5A-5C, which represent CT slices of the unenhanced (FIG. 5A), arterial (FIG. 5B) and excretory (FIG. 5C) phases. In FIG. 5D, the urinary excretion of contrast media into the kidney tubules (light gray area) was: the volume of both kidneys multiplied by mean attenuation of kidneys at the excretory phase minus mean attenuation of kidneys at the unenhanced phase, then divided by time period: 257.5× (81.1−35.9)/9.57=1216 HU×mL/min. In FIG. 5E: the urinary excretion of the contrast agent into the kidney tubules was the sum of urinary excretion of the contrast agent into the kidney medulla (light gray area) and urinary excretion of the contrast agent into the kidney cortex (dark gray area). The urinary excretion of the contrast agent into the kidney cortex was the volume of kidney cortex multiplied by mean CT-attenuation of kidney at the excretory phase minus CT-attenuation of the kidneys in the unenhanced phase, and minus cortical CT attenuation from the vascular compartment in the excretory phase (i.e. ratio between the enhancement of the cortex and that of the aorta during the arterial phase, then multiplication of this ratio by the aorta contrast uptake in the excretory phase) then divided by time between arterial and excretory phases: 148.4×(81.1−35.9−(489.6−35.9)/(943.9−40.3)×(76.6−40.3)/9.57=418 HU×mL/min. The urinary excretion of the contrast agent into the kidney medulla was the volume of kidney medulla multiplied by mean CT-attenuation of kidneys at the excretory phase minus mean attenuation of kidneys at the unenhanced phase, then divided by time period: (257.5−148.4)×(81.1-35.9)/9.57=515 HU×mL/min. The urinary excretion of the contrast agent into the kidney tubules was therefore: 418+515=933 HU×mL/min.

FIGS. 6A-6B: Bland-Altman plot showing agreement between CT-measured GFR and mGFR. The x-axis shows the mean of the GFR measurements obtained by the two assessment methods. The y-axis shows the relative difference between the GFR measurements from the two assessment methods. The solid line is the bias (the mean relative difference), and the dashed lines are the lower and upper limits of the interval of agreement (−1.96 SD and +1.96 SD). The black dots are the 28 included chronic kidney disease (CKD) patients. White dots are the 75 previously reported healthy individuals. In FIG. 6A, the CT-mGFR is calculated assuming that the contrast media in the kidney parenchyma during the excretion phase is located exclusively in the tubular compartment. In FIG. 6B, the part of kidney cortex enhancement in the excretory phase attributable to the vascular compartment of the cortex was subtracted from CT-mGFR calculation.

FIGS. 7A-7B: Bland-Altman plot showing of interobserver agreement between CT-mGFR determined by a senior nephrologist with 8 years experience in GFR measurement, and CT-mGFR determined by a senior radiologist with 5 years experience in abdominal radiology, in CKD patients. Each observer was blinded to the result obtained by the other as well as to the result of GFR measurement by iohexol clearance. In FIG. 7A, the CT-measured GFR is calculated as previously published, assuming that the contrast media in the kidney parenchyma during the excretory phase is located exclusively in the tubular compartment. In FIG. 7B, the part of kidney cortex enhancement in the excretory phase attributable to the vascular compartment of the cortex was subtracted from CT-measured GFR calculation.

### EXAMPLES

### Example 1: Iomeprol Clearance Assessed by CT Urography to Measure GFR in Living Kidney Donor Candidates

### Methods

### Study Design:

This is a cross-sectional study using data from kidney donor candidates who were screened in hospital between July 2016 and October 2022, with both GFR measurement by iohexol clearance, and kidney morphology assessment by CT urogaphy.

The Institutional Review Board of our Institution approved this study: CSE-21-23_PEGMAS, and the patients gave their informed consents.

### Study Population:

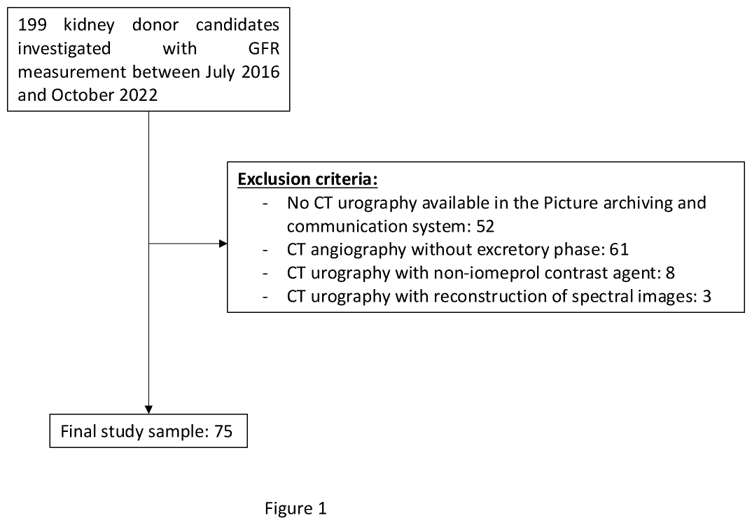

All living kidney donor candidates investigated in Nephrology Department between July 2016 and October 2022, were eligible for inclusion. In accordance with the KDIGO guidelines (Lentine K L et al. ([11])), GFR was measured in all these individuals from iohexol clearance. The only inclusion criterion was to have had a CT urography during the same period, available in the hospital's Picture archiving and communication system (whether the CT scan was performed in the hospital or outside). The CT urography had to include 4 polychromatic acquisition phases (unenhanced, arterial, nephrographic, and excretory phases) with a monophasic injection of iomeprol at 350 or 400 mg of iodine per milliliter (Iomeron® 350 or 400, Bracco Imaging, Milano, Italy). The flow chart is presented in FIG. 1.

### Clinical and Biological Data

Sex, age, weight, height, and serum creatinine values were collected from medical records.

GFR Measurement with Iohexol Clearance:

After a 5 mL bolus injection of iohexol (300 mg/L Omnipaque®; GE Healthcare, France), and an equilibration period (time the tracer takes to distribute in the extracellular compartment), blood and urine samples were collected over four to six consecutive clearance periods. We performed six 30-min periods after a 90-min equilibration period until July 2019, then four 40-min periods after a 120-min equilibration. The concentrations of iohexol in serum and urine were determined by high-performance liquid chromatography (HPLC) as previously described (Cavalier E et al. ([12])). Measured GFR (mGFR) was the mean of the four to six clearance-period values calculated as follows: U×V/P, where U is the concentration of iohexol in the urine collected during the period, V is the urinary flow rate during the period, and P is the serum concentration of iohexol at mid period. Given the negative bias of this GFR measurement method (Seegmiller J C et al. ([13]), Stehlé T et al. ([14])), the following correction was used: mGFR=1.15×iohexol urinary clearance+1.3 (Stehlé T et al. ([14])). If voiding was irregular or incomplete or in case of urine loss, mGFR was calculated with iohexol plasma clearance determined from the plasma disappearance curve, with Bröchner-Mortensen correction model for the missing early compartment (Bröchner-Mortensen J. A ([15])).

GFR Measurement with Iomeprol Clearance Assessed by CT Urography (CT-mGFR):

CT-mGFR was assessed by the same U×V/P calculation used to determine the urinary clearance of iohexol: the amount of iomeprol excreted through the urinary system during a given period of time was divided by the mean serum iomeprol concentration during the same period. The blood and urine concentration of iomeprol were assessed by attenuation measurement (Hounsfield Units, HU) in the different areas of interest. The time periods were derived from the acquisition time of the different phases. These data were retrieved for all patients and phases in the DICOM 0008-0032 “Acquisition Time” header, which represents the time when the acquisition started. Manual segmentations of bladder, upper urinary tracts and kidneys were performed using the Advantage Windows software (Advantage Window v4.7; GE Healthcare, Buc, France), using different thresholds depending on the CT phase and then manually finalized. For non-contrast and portal venous phase: only a lower limit was set to 0 HU, in order to exclude fat pixels. For excretory phases: a specific threshold was adapted for each patient based on visual analysis in order to reduce extra-urinary pixels as much as possible. Also, the lower threshold used for upper excretory tract was used as the higher threshold for kidney segmentation to avoid overlap in segmentations. The urinary excretion rate of iomeprol between arterial and excretory phases (U×V, expressed as HU/min) was calculated from urinary excretion of iomeprol into the bladder (Uexcr-Bladder), urinary excretion of iomeprol into the upper excretory tract (calyces, pelvis, ureters) (Uexcr-tract), and urinary excretion of iomeprol into the kidney tubules (Uexcr-tubules): U×V=Uexcr-Bladder+Uexcr-tract+Uexcr-tubules. Uexcr-bladder was the volume of the bladder at the excretory phase (Bladder-Volume) multiplied by the mean CT-attenuation of the bladder at the excretory phase (HU-Bladder-excr) minus the CT-attenuation of the urine in bladder at the unenhanced phase (HU-Bladder-unenhanced), then divided by the time period between the excretory phase and the arterial phase (time period): Uexcr-Bladder=Bladder-volume×(HU-Bladder-excr−HU-Bladder-unenhanced)/time period. When the bladder was not included on the unenhanced phase, urine CT-attenuation could be measured on the nephrographic phase, the urine density at the nephrographic phase being slightly higher than at the unenhanced phase, but without clinical consequence on the CT-mGFR calculation. Uexcr-tract was determined in the same way by studying the upper excretory tract (calyces, pelvis, ureters) instead of the bladder. Uexcr-tubules was the volume of both kidneys (Kidney-volumes) multiplied by mean CT-attenuation of kidneys at the excretory phase (HU-Kidney-excr) minus mean CT-attenuation of kidneys at the unenhanced phase (HU-Kidney-unenhanced), then divided by time period: Uexcr-tubules=Kidney-volumes×(HU-Kidney-excr-HU-Kidney-unenhanced)/time period. The denominator of the U×V/P equation, was the calculated serum iomeprol concentration at the midpoint of the period between the arterial and excretory phases. At each phase, the difference between the CT attenuation of the aorta and the one at the unenhanced phase was the surrogate for the serum iomeprol concentration. By taking into account the aortic CT-attenuation at the unenhanced phase, the hematocrit was considered in the measurement of iomeprol, thus providing an equivalent of serum assay rather than a whole blood assay (Black D F et al. ([16])). Because the decrease in serum iomeprol concentration during the period between arterial and excretory phases (which includes both its distribution in the extracellular compartment and its urinary excretion by glomerular filtration) follows an exponential curve, this decrease was modeled by linear regression after transforming each serum concentration value equivalent (in HU) into natural logarithm values. The serum concentration at half time was deduced from this linear regression. Each measurement was performed by one senior radiologist. An example of CT-mGFR calculation from a patient is provided below (Step-by-step description of the CT-mGFR calculation), and an online calculator is also available at the following web address: https://paul-bssr-app-streamlit-gfr-streamlit-app-6uzfvr.streamlit.app/:

Step-by-Step Description of the CT-mGFR Calculation: Example Using Values from a Patient's CT Scan

The equation to calculate CT-mGFR is:

$$( Uexcr - Bladder ( A ) + Uexcr - tract ( B ) + Uexcr - tubules ( C ) ) / Iomeprol serum concentration at mid - period ( D )$$

A) Uexcr-Bladder: Excretion of Iomeprol into the Bladder
3D segmentation of the bladder at the unenhanced phase: Mean attenuation 23.9 HU 3D segmentation bladder at the excretory phase: Volume 161 mL, Mean attenuation 445.6 HU
1. Volume of the bladder at the excretory phase: 161 mL
2. Time between the arterial phase and the excretory phase: 9.47 min
3. Mean CT-attenuation of the bladder at the excretory phase (HU-Bladder-excr): 445.6 HU
4. Mean CT-attenuation of the urine in bladder at the unenhanced phase (HU-Bladder-unenhanced): 23.9HU

$$\text{?} Uexcr - Bladder = ( 445.6 - 23.9 ) \times 161 / 9.47 = 7169.3 HU \times mL / min$$

B) Uexcr-Tract: Urinary Excretion of Iomeprol into the Upper Excretory Tract (Calyces, Pelvis, Ureters)
3D segmentation of the bladder at the unenhanced phase: Mean attenuation 23.9 HU 3D segmentation of the upper tract at the excretory phase: Volume 15.7 mL, Mean attenuation 738.2 HU
1. Volume of the excretory tract at the excretory phase: 15.7 mL
2. Time between the arterial phase and the excretory phase: 9.47 min
3. Mean CT-attenuation of the excretory tract at the excretory phase (HU-excr-tract): 738.2 HU
4. Mean CT-attenuation of the urine in bladder at the unenhanced phase (HU-Bladder-unenhanced): 23.9HU

$$\text{?} Uexcr - tract = ( 738.2 - 23.9 ) \times 15.7 / 9.47 = 1184.2 HU \times mL / min$$

C) Uexcr-Tubules: Urinary Excretion of Iomeprol into the Kidney Tubules
3D segmentation of the kidneys at the unenhanced phase: Volume 387 mL, Mean attenuation 34.8 HU
3D segmentation of the kidneys at the excretory phase: Mean attenuation 97.7 HU
1. Volume of both kidneys: 387 mL
2. Time between the arterial phase and the excretory phase: 9.47 min
3. Mean CT-attenuation of the kidneys at the excretory phase (HU-Kidney-excr): 97.7 HU
4. Mean CT-attenuation of the kidneys at the unenhanced phase (HU-Kidney-unenhanced): 34.8 HU

$$\text{?} Uexcr - tubules = ( 97.7 - 34.8 ) \times 387 / 9.47 = 2570.5 HU \times mL / min$$

### D) Iomeprol Serum Concentration at Mid-Period Between the Arterial and Excretory Phases

Aorta CT-attenuation at the unenhanced phase: 46 HU
Aorta CT-attenuation at the arterial phase: 256.2 HU
Aorta CT-attenuation at the nephrographic phase: 134.3 HU
Aorta CT-attenuation at the excretory phase: 92.8 HU
   1. Serum iomeprol concentrations assessed at the arterial phase=256.2−46=210.2 HU
   2. Serum iomeprol concentrations assessed at the nephrographic phase=134.3−46=88.3HU
   3. Serum iomeprol concentrations assessed at the excretory phase=92.8−46=46.8 HU
   4. Natural logarithm transformation of the 3 serum iomeprol concentration values
      i. Ln (Serum iomeprol concentrations assessed at the arterial phase)=Ln (210.2)=5.35
      ii. Ln (Serum iomeprol concentrations assessed at the nephrographic phase)=Ln (88.3)=4.48
      iii. Ln (Serum iomeprol concentrations assessed at the excretory phase)=Ln (46.8)=3.85
   5. Determine the equation of the linear regression of these 3 logarithmic values as a function of time (For this patient: arterial phase: 0 min, nephrographic phase: 0.77 min and Excretory phase: 9.47 min): Ln (iomeprol)=4.98−0.123×time (min)
   6. Determine the serum concentration (in HU) of iomeprol at mid-period between the arterial and excretory phases: Ln (iomeprol mid-period)=4.98−0.123×9.47/2=4.396

$$\text{?} Iomeprol mid - period = Exp ( 4.396 ) = 81.1 HU$$

### Calculation of CT-mGFR:

In this example the CT-mGFR is therefore:

$$( Uexcr - Bladder ( A ) + Uexcr - tract ( B ) + Uexcr - tubules ( C ) ) / Iomeprol mid - period ( D ) = ( 7169.3 + 1184.2 + 2570.5 ) / 81.1 = 134.2 ml / min$$

CT-mGFR is then adjustable to the body surface area (BSA):

The patient weighs 99 kg and is 175 cm in height=>BSA is 2.18 m² according to the Mosteller formula

$$CT - mGFR = 134.2 / BSA \times 1.73 = 106. ml / min / 1.73 {m}^{2}$$

### Statistical Analysis

Continuous variables were expressed as median and interquartile ranges (IQR) or mean and standard deviation (SD), as appropriate. We analyzed the relationships between CT-mGFR and iohexol urinary clearance by Passing-Bablok regression, calculating the slope, intercept, and their 95% confidence intervals (95% CI) (Passing H et al. ([17]). We evaluated the performance of CT-mGFR, compared with mGFR, by determining mean bias as the mean difference between CT-mGFR and mGFR, precision as the standard deviation of the bias, and accuracy as the percentage of CT-mGFR falling within 10%, 20% and within 30% of mGFR. We provided visual representation of the agreements with Bland Altman plots (Bland J M, Altman D G ([18])). Agreement between GFR determination methods were also assessed by Lin's concordance correlation coefficient (CCC) (Lin L I ([19])). The same analyses were performed to assess the performance of eGFR determined from the CKD-EPI₂₀₂₁ equation (Inker L A ([20])), with plasma creatinine routinely measured on the same time as iohexol clearance. We compared the precisions of CT-mGFR vs. CKD-EPI₂₀₂₁ using the Pitman test for comparison of variances of correlated samples. To compare accuracies, we used the McNemar test. Statistical analyses were conducted with Microsoft® Excel and XLSTAT® software (Addinsoft 2021).

### Results

### Clinical Characteristics:

Among 199 kidney donor candidates who underwent GFR measurement with iohexol clearance between July 2016 and October 2022, 75 were included. The main reasons for non-inclusion were no CT urography available on the Picture archiving and communication system (n=52), or CT scans performed without excretory phase (n=61) (FIG. 1). The demographic and morphometric characteristics of the subjects are described in Table 1.

TABLE 1
Description of study population
Study population clinical characteristics:

| Age, years, mean ± SD | 50.46 ± 13.0 | |
|---|---|---|
| Female, N (%) | 45 | (60) |
| African or Caribbean ancestry, N (%) | 20 | (27) |
| Body weight, Kg, mean ± SD | 73.1 ± 15.8 | |
| Height, cm, median [IQR] | 166 | [160-173] |
| BMI, Kg/m2, mean ± SD | 25.8 ± 3.8 | |
| Time between GFR measurement and CT scan, | | |
| days, median [IQR] | 7 | [−12.5-23.5] |

| | |
|---|---|
| mGFR, ml/min, mean ± SD | 99.7 ± 19.1 |

SD, standard deviation; IQR, interquartile range.

### CT Urography Protocols:

Details of the CT urography instrumentation are provided on Table 2.

TABLE 2
CT urography protocols and CT-scan
data used to calculate CT-mGFR
CT urography protocols (data not included
in the calculation of CT-mGFR):

| | |
|---|---|
| n = GE Revolution CT ®, n = GE Discovery | 22/48/5 |
| CT ®, n = others | |
| X-ray tube voltage, n = 80 kVp/n = | 2/13/58/1 |
| 100 kVp/n = 120 kVp/n = 140 KkVp | |
| Slice thickness, n = 1.25 mm/n = 2.0 mm | 73/2 |
| Iomeprol dose injected, mg/Kg, mean ± SD | 948.5 ± 171.4 |
| Iodine dose injected, mg/Kg, mean ± SD | 465.0 ± 83.8 |

CT urography protocols (data included in
the calculation of CT-mGFR)

| Time between arterial and nephrographic | 0.78 | [0.74-0.83] |
|---|---|---|
| phases, minutes, median [IQR] | | |
| Time between arterial and excretory | 9.5 | [9.3-9.7] |

phases, minutes, median [IQR]

CT urography data used to calculate CT-mGFR

| CT-attenuation value of the aorta at the | 41.2 ± 6.2 | |
|---|---|---|
| unenhanced phase, HU, mean ± SD | | |
| CT-attenuation value of the aorta at the | 311.2 ± 83.0 | |
| arterial phase, HU, mean ± SD | | |
| CT-attenuation value of the aorta at the | 150.2 ± 26.4 | |
| nephrographic phase, HU, mean ± SD | | |
| CT-attenuation value of the aorta at the | 86.9 ± 13.6 | |
| excretory phase, HU, mean ± SD | | |
| Total volume of the 2 kidneys, mean ± SD | 309.5 ± 66.1 | |
| CT-attenuation value of the kidneys at | 32.9 ± 3.6 | |
| the unenhanced phase, HU, mean ± SD | | |
| CT-attenuation value of the kidneys at | 116.5 ± 17.1 | |
| the excretory phase, HU, mean ± SD | | |
| CT-attenuation value of the bladder at | 14.2 | [10.1-21.2] |
| the unenhanced phase, HU, median [IQR] | | |
| Volume of the bladder at the excretory | 110 | [75.7-165.5] |
| phase, mL, median [IQR] | | |
| CT-attenuation value of the bladder at | 363.6 | [264.0-552.9] |

| | |
|---|---|
| the excretory phase, HU, median [IQR] | |
| Volume of the upper excretory tract at | 16.3 ± 6.2 |
| the excretory phase, mL, mean ± SD | |
| CT-attenuation value of the excretory | 1053.7 ± 317.9 |
| tract at the excretory phase, HU, | |
| mean ± SD | |

kVp, kilovoltage peak; SD, standard deviation; IQR, interquartile range; HU, Hounsfield unit.

Seventy CT urography were performed in our department on a GE Discovery CT® (n=48) or on a GE Revolution CT® (n=22), and 5 CT urography were performed externally, with Siemens SOMATOM Definition AS® (n=1), Toshiba Aquilion PRIME® (n=1), GE optima CT540® (n=2), or Philipps Ingenuity CT® equipment (n=1). The X-ray tube voltage was mainly 120 kVp, with the same x-ray tube voltages between the different acquisitions, and the slice thicknesses 1.25 mm. Iterative reconstructions were applied for 64 patients and TrueFidelity Deep Learning Image Reconstruction (TF-H GE®) for 11. Soft filter kernel was applied to all acquisitions. Except for 7 patients who received iomeprol 400 mg/L, all received iomeprol 350 mg/L. The mean administered dose of iomeprol was 948.5±177.4 mg/Kg (Table 2), with extreme dosages from 676.4 mg/Kg to 1350 mg/Kg. The iomeprol injection rate was between 2.5 and 3.5 ml/sec depending on the quality of the venous access, except for CT urography performed externally, in which the injection rate was not known (n=5). Bolus tracking software (Smartprep, GE Healthcare, WI, USA) was used for the 68 CT performed at our institution with an acquired arterial phase 20 sec (n=30) or 6 sec (n=38) after the attenuation increase in abdominal aorta reached the predefined thresholds of 100 HU and 250 HU, respectively. For the remaining 5 patients, the delay of arterial phase was not available. Only two of the patients in the study received furosemide. Median time between the arterial and nephrographic phases, recovered from DICOM data, was 47 seconds [IQR: 44; 50] (min: 31; max: 74). Median time between the arterial and the excretory phases was 9.5 minutes [IQR: 9.3; 9.7] (min: 6.2; max 13.3).

### GFR Measurement Using Iomeprol Clearance Assessed by CT Urography (CT-mGFR):

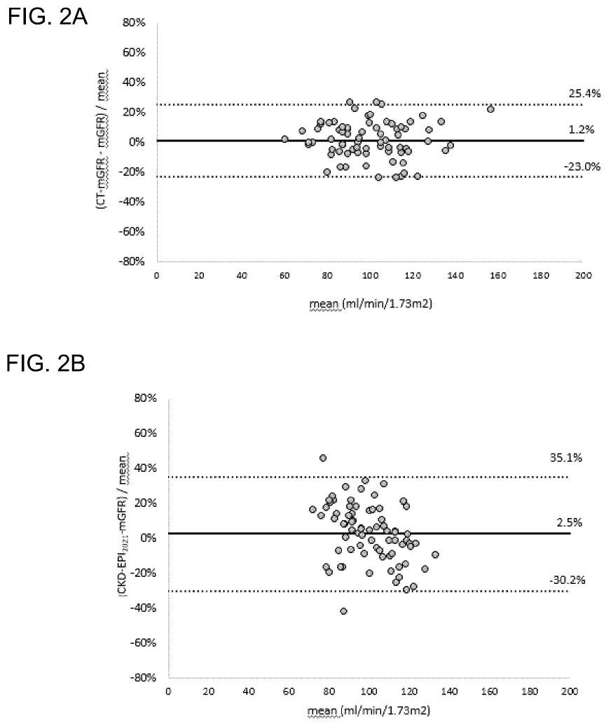

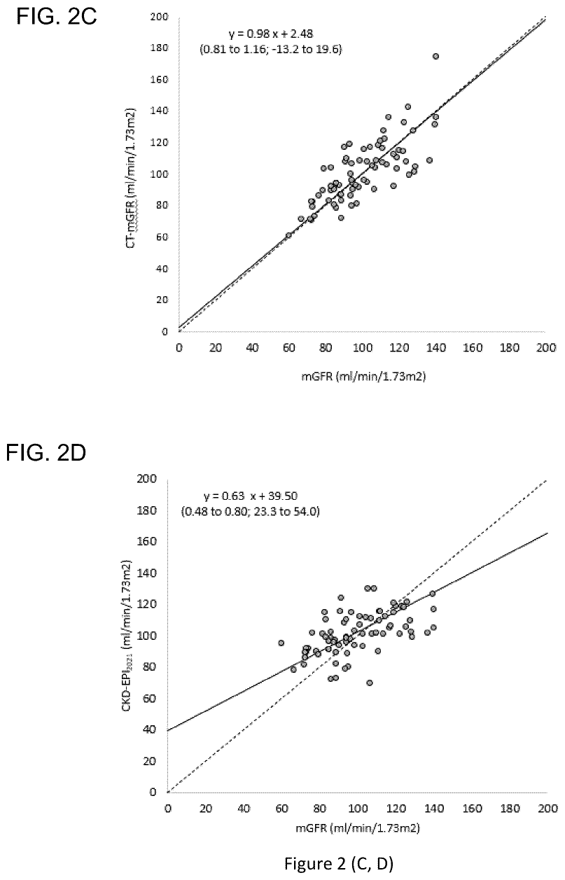

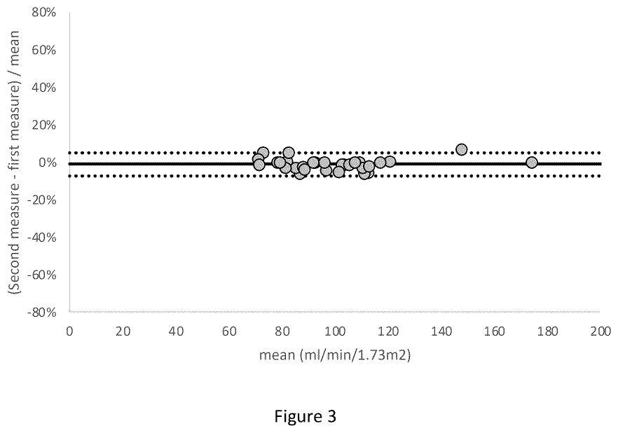

The mean and median values of the different CT-attenuations and volumes, used to calculate the CT-mGFR are described in Table 2. Mean CT-mGFR was 100.9±19.5 ml/min/1.73 m² (min=60.8, max=174.0), not statistically different from mGFR which was 99.7±19.1 ml/min/1.73 m² (min=59.9, max=140.2) (paired t-test, p-value: 0.47). The relationship between CT-mGFR and mGFR obtained by Passing-Bablok regression is illustrated in FIGS. 2A-2D: the regression line was very close to the equivalence line. The agreement between CT-mGFR and mGFR is also depicted as a Bland Altman plot (FIGS. 2A-2D): the mean bias was 1.1 ml/min/1.73 m² (−1.9; 4.1) (table 3). The accuracy within (AW) 10%, 20%, and 30% was 61.3% (95% Cl: 50.3; 72.4), 88.0% (95% Cl: 80.7; 95.4), and 100%, respectively.

TABLE 3
Assessing the performance of CT-mGFR and CKD-EPI₂₀₂₁, relative to mGFR

| | Absolute | IQR of | | | | |
|---|---|---|---|---|---|---|
| | bias, mean | the bias | Accuracy | Accuracy | Accuracy | |
| | (95% CI) | [Q1; Q3] | Within | Within | Within | |
| | (ml/min/ | (ml/min/ | 30% | 20% | 10% | Lin's CCC |
| | 1.73 m2) | 1.73 m2) | (95% CI) | (95% CI) | (95% CI) | (95% CI) |
| CT-mGFR | 1.1 | 16.2 | 100% | 88.0% | 61.3% | 0.766 |
| vs mGFR | (−1.9; 4.1) | [−6.6; 9.6] | | (80.7; 95.4) | (50.3; 72.4) | (0.653; 0.845) |
| CKD-EPI₂₀₂₁ | 1.6 | 21.6 | 92.0% | 77.3% | 44.0% | 0.504 |
| vs mGFR | (−2.2; 5.3) | [−8.8; 12.8]* | (85.9; 98.1)* | (67.9; 86.8) | (32.5; 55.6)* | (0.330; 0.644)* |

95% CI, 95% confidence interval; IQR, interquartile range; Q1, quartile 1; Q3, quartile 3; Lin's CCC, Lin's concordance correlation coefficient.
P-values were calculated between CT-mGFR and CKD-EPI₂₀₂₁.
*P < .05.
The precision (IQR of the bias) comparison was performed with Pitman's test.
Accuracy comparison was performed with McNemar's test.

The accuracy of CT-mGFR was not affected by patient ethnicity or hydration status (data not shown), and did not appear to be obviously affected by the dose of iomeprol administered (data not shown), the model or brand of CT scan (data not shown), the time interval between the arterial and excretory phases (data not shown), the CT tube voltage (data not shown), the CT reconstruction modality (data not shown), or the acquisition procedure for the arterial phase (data not shown). The two patients who received furosemide had reasonably good results (CT-mGFR was-8% and +12% of mGFR).

CT-mGFR had better precision and accuracy than CKD-EPI₂₀₂₁ (Table 3, FIG. 2).

### Intra-Observer Reproducibility for CT-mGFR:

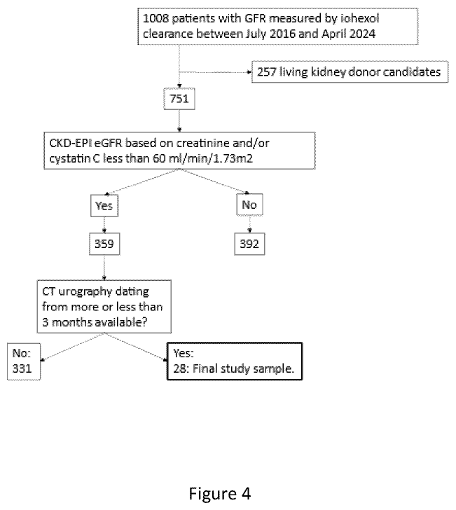

Intra-observer reproducibility for CT-mGFR were determined from 30 randomly selected patients from the study population. Bland Altman (FIG. 4) illustrate unbiased pairwise measures with excellent agreements. Accuracy within 10% was 100% and Lin's concordance correlation coefficient was 0.989 (95% Cl: 0.978; 0.994).

### Discussion

Based on the results of our study, we proposed a novel method for measuring GFR using the measurement of a contrast agent clearance, such as iomeprol, assessed by CT urography (CT-mGFR), allowing accurate and reproducible assessment of GFR in living kidney donor candidates.

It was proposed that GFR measurement methods be considered to have sufficient accuracy when median bias was less than 5% compared to the reference method, with at least 80% of the measurements within ±30% of the reference measurements, and at least 50% within ±10% (Soveri I et al. ([21])). Iomeprol is a nonionic, hydrosoluble, iodinated contrast agent with low viscosity and low osmolality. Its molecular weight of 777.09 Dalton is slightly lower than that of iohexol (821.1 Da) or iothalamate (809.1 Da), the two iodinated contrast agents that are widely used for GFR measurement. Furthermore, iomeprol does not bind measurably to plasma proteins (Lorusso V et al. ([22]). These chemical properties make iomeprol potentially an ideal exogenous marker for measuring GFR, i.e. it would diffuse freely into the extracellular volume, not be metabolized nor bound to plasma proteins, be eliminated exclusively by the kidney, as it would be freely filtered, and not secreted or reabsorbed in the tubule. Pharmacokinetic studies confirmed that after a bolus injection of iomeprol, its plasma concentration declined biexponentially (first slope corresponding to its diffusion in the extracellular volume and its renal clearance, and second slope corresponding only to its renal clearance), with a negligible extra-renal clearance, and a very good correlation between its clearance and that of inulin (Lorusso V et al. ([23]).

Our method of CT scan-based GFR measurement is accurate, and is usable with CT urography data obtained from routine care, in an opportunistic value-added CT imaging approach, provided that unenhanced, arterial, nephrogenic, and excretory acquisition phases are available. Because CT-mGFR is a measure of clearance of an exogenous tracer (iomeprol), it is accurate regardless of patient origin. In our population, there was some variability from one patient to another for the dose of iomeprol injected, which did not prevent our method from being efficient, since urinary clearance measurement of an exogenous tracer is not dependent on the dose of tracer administered. The variability of acquisition times for excretory phase, from one patient to another, did not interfere with the effectiveness of our method either: when measuring the urinary clearance of an exogenous tracer, what is crucial is not to respect a very precise timing, but to record very precisely this sampling timing, which is done in an automated way during a CT-scan, these image acquisition times are recorded in the DICOM headers. CT-scan thereby avoids potential inaccuracies related to errors in recording blood or urine collection times, as it can happen in standard GFR measurement methods. Also, there is no inaccuracy related to deficient bladder emptying, or uncollected urine (which is also a major limitation of the accuracy of GFR measurement with urinary clearance procedures). The CT-mGFR also has the advantage of being much shorter in time and less cumbersome than a GFR measured with exogenous tracer clearance. Moreover, it is without additional cost if it is performed in patients having a CT-scan as part of their medical follow-up, such as kidney donor candidates, or some pre-cancer nephrectomy assessments for example. Kidney volumetry and kidney enhancement could also provide information on the distribution of function between the 2 kidneys, an information that is not provided by the GFR measurement based on exogenous tracer clearance. Lastly, we note that the variability from one patient to another for CT scan brands and models, for CT tube voltages, and for reconstruction modalities, did not seem to influence the performance of our measure.

In conclusion, it is possible to measure GFR accurately from CT urography performed in the routine care setting, without any specific acquisition protocol other than performing unenhanced, arterial, nephrographic or portal venous, and excretory phases.

### Example 2: Contrast Media Clearance Assessed by CT Urography to Measure GFR in Subjects Having Low GFR

For patients with chronic kidney disease (i.e. estimated GFR<60 ml/min/1.73 m², and/or other features of chronic kidney disease) a particular embodiment of the method of the invention may be performed in order to avoid or reduce overestimation of the measured GFR compared to healthy individuals.

Indeed, overestimation averaged 12% in the patients having chronic kidney disease.

The inventors showed that this is due to the fact that in calculating the rate of urinary excretion of the contrast product between the arterial and excretory phases, the tubular component is overestimated.

Thus, they developed an embodiment of the method of the invention more adapted for patients having low GFR, comprising a segmentation of the cortex. This method, which has a minimal impact in healthy individuals, can also be used in them.

### Method

### Study Sample:

This retrospective cross-sectional study used data from all the patients who had a GFR measurement by iohexol clearance between July 2016 and April 2024 in our center, in whom the creatinine-based eGFR and/or the cystatin C-based eGFR was below 60 ml/min/1.73 m², and who had a CT urography within an interval of less than 3 months around the GFR measurement. CT urography had to be available on the hospital picture archiving and communication system and had to include the following 4 phases: unenhanced, arterial, nephrographic, and excretory phases with the same X-ray tube voltages between the different acquisitions. There were no exclusion criteria. The performance of GFR measured by CT urography in CKD patients was compared to that of GFR measured by the same method in the 75 healthy individuals from the original study. The French Ethics Committee for the Research in Medical Imaging (CERIM) institutional review board approved this study (no. CRM-2403-402) and patients gave informed consent.

GFR Measurement with Iohexol Clearance:

Iohexol clearance was measured as previously described in the example 1.

GFR Measurement with Contrast Media Clearance Assessed at CT Urography:

A senior nephrologist performed the segmentations of CKD patients and healthy individuals using 3D slicer 5.4.0 software. For the assessment of interobserver reproducibility in CKD patients, the senior radiologist who had previously measured GFR from CT urography in healthy individuals also determined the CT-measured GFR of CKD patients, using Advantage Windows software (version 4.7; GE Healthcare) as in example 1. Interobserver agreement at normal GFR ranges, which had not been previously evaluated, was determined using the CT-measured GFR values previously calculated in living kidney donors by the senior radiologist.

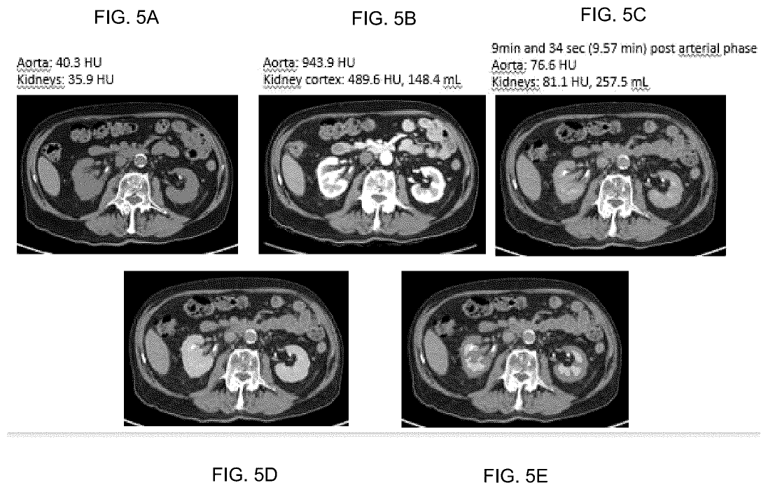

CT-measured GFR was calculated as in the example 1. We also evaluated a CT-measured GFR calculation in which the part of kidney cortex enhancement in the excretory phase attributable to the vascular compartment of the cortex was subtracted. The details of the required segmentations and calculation steps of this modified method for determining tubular excretion rate of contrast media are provided in FIGS. 5A-5E.

### Statistical Analysis

Continuous variables were expressed as medians with interquartile ranges (IQRs) or means±standard deviations (SDs), as appropriate. We evaluated the performance of CT-measured GFR based on: 1/the relative bias defined as the mean of the difference between CT-mGFR and measured GFR divided by measured GFR (mGFR), 2/the precision, defined as the distance between quartile 1 and quartile 3 of the bias, 3/the accuracy defined as the percentage of patients with CT-measured GFR falling within 10%, 20% and 30% of mGFR. Bland-Altman plots were used to illustrate the agreement between CT-measured GFR and mGFR. The interobserver agreement was also evaluated using the Lin concordance correlation coefficient. Precision of CTmGFR in CKD patients was compared to that of CTmGFR in healthy individuals using the F test for comparison of variances, and accuracy was compared using the Fisher exact test. In the CKD patient group, and then in the healthy individual group. P<0.05 was considered indicative of statistically significant difference. Statistical analyses were conducted with Microsoft Excel and XLSTAT software (2024.1.0 version, Addinsoft).

### Results

### Characteristics of CKD Patients

The study included 28 patients (FIG. 4), 10 females and 18 males. The mean age was 68.7 years±16.4, and 5 patients were between 85 and 90 years old. Mean BMI was 25.5 kg/m2±4.2, with 4 obese patients (BMI>30). The most frequent reason for measuring GFR was to predict post-surgical GFR in patients with CKD who were to undergo nephrectomy or nephroureterectomy for cancer, in order to predict post-surgical GFR. Mean GFR based on iohexol clearance was 46.0 ml/min/1.73 m²±15.8. Complete characteristics of the patients are provided on table 4.

### CT Urography Parameters

All but one of the CT scans were performed on GE HealthCare revolution equipment. For 22 patients, CT tube voltages ranged from 100 to 120 kVp. Six patients had dual-energy CT urography (80-140 KVp) with reconstruction of monochromaticimages at 40 KeV. Slice thickness was 1.25 for 26 of 28 patients. The iodinated contrast agent was iomeprol for 18 patients, iobitridol for 6 patients, iodixanol for 2 patients, and was unknown for 2 patients who underwent CT urography at an external facility (Table 4).

TABLE 4
Characteristics of the study population
and CT urography parameters

| Variables | Values | |
|---|---|---|
| No. of patients | 28 | |
| Age (years)* | 68 .7 ± 16.4 | |
| Sex (F/M) | 10/18 | |
| Body weight (Kg)* | 74.0 ± 15.8 | |
| Height (cm)* | 169.7 ± 10.2 | |
| Body mass index (Kg/m²)* | 25.5 ± 4.2 | |
| Iohexol clearance-measured | 46.0 ± 15.8 | |
| GFR (mL/min/1.73 m2)* | | |
| Clinical indication for CT scan | | |
| Kidney cancer or urothelial | 14/28 | (50%) |
| cancer diagnosis | | |
| Non-confirmed suspicion of | 2/28 | (7%) |
| kidney cancer or urothelial cancer | | |
| Investigating the cause of hematuria | 5/28 | (17%) |
| Post cancer nephrectomy monitoring | 4/28 | (14%) |
| Morphological assessment in kidney stone | 2/28 | (7%) |
| disease | | |
| Investigating unilateral kidney atrophy | 1/28 | (4%) |
| Clinical indication for GFR | | |
| measurement with iohexol clearance | | |
| Prediction of post-nephrectomy GFR | 14/28 | (50%) |
| confirmation of chronic kidney | 4/28 | (14%) |
| disease (low eGFR, but no other | | |
| Dose adjustment of drugs with a narrow | 3/28 | (11%) |
| therapeutic margin | | |
| Symptoms suggestive of severe CKD | 7/28 | (25%) |
| while the eGFR is [symbol] | | |
| Days between GFR measurement and CT scan ^{†} | 4 | [−10.5; 11.8] |
| CT system | | |
| GE Revolution ™ | 27/28 | (96%) |
| SIEMENS SOMATOM ® go.Top | 1/28 | (4%) |
| Tube voltage | | |
| 100 kVp | 8/28 | (29%) |
| 110 kVp | 1/28 | (4%) |
| 120 kVp | 13/28 | (46%) |
| 80-140 kVp (Dual-energy spectral CT) | 6/28 | (21%) |
| Section thickness | | |
| 1.25 mm | 26/28 | (93%) |
| 1.5 mm | 1/28 | (4%) |
| 2 mm | 1/28 | (4%) |
| Contrast media agent | | |
| iomeprol | 18/28 | (69%) |
| lobitridol | 6/28 | (21%) |
| iodixanol | 2/28 | (7%) |
| Unknown | 2/28 | (7%) |

| | |
|---|---|
| Iodine dose injected (mg/Kg)* | 455.5 ± 110.0 |

Notes:
*Data are means ± SDs.
^{†}Data are medians, with IQRs in Body mass index is patient weight in kilograms divided by patient height in meters [symbol]
indicates data missing or illegible when filed

### Comparison of GFR Measured Using Contrast Media Clearance Assessed at CT Urography and Iohexol Clearance

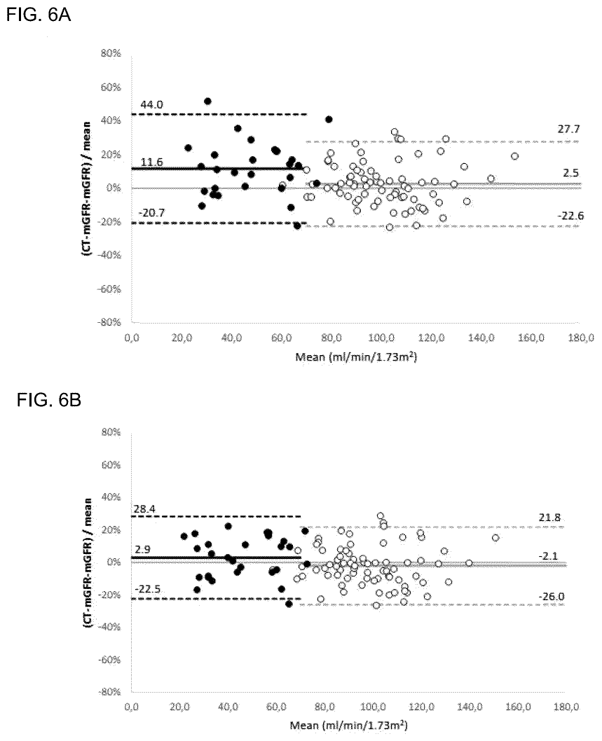

When the CT-mGFR was calculated on the assumption that all kidney parenchymal enhancement in the excretory phase resulted from the tubular compartment, it overestimated the mGFR calculated by iohexol clearance by 11.6% (95% Cl: 5.5, 17.7) (Table 5, FIGS. 6A-6B). In comparison with the CT-mGFR calculated in living kidney donor candidates, which was unbiased (mean bias 2.4% (95% Cl: −0.05, 5.3) and accurate (Accuracy within 20%: 85.3 (95% Cl: 77.3, 93.3)), the CT-mGFR of CKD patients was less accurate (Accuracy within 20:67.9% (95% Cl 50.6, 85.2), P values: 0.06 in a context of limited statistical power due to the small number of patients). When the part of kidney cortex enhancement in the excretory phase attributable to the vascular compartment of the cortex was subtracted from the calculation of the CT-mGFR, the CT-mGFR became unbiased (mean bias: 2.9% (−1.9, 7.7)), with accuracies within 30, 20 and 10% that became very high at 100%, 92.9% (95% Cl: 83.3, 100) and 50% (95% Cl 31.5, 68.5), not statistically different from those found in healthy subjects (100%, 89.3% (95% Cl: 82.4, 96.3), and 54.7 (95% Cl: 43.3, 65.9), P values 1, 0.6, 0.7, respectively). The impact of subtracting the cortical enhancement from the vascular compartment on the calculation of CT-mGFR in healthy individuals was non-significant, with no bias vs iohexol clearance (mean bias: −2.2% (95% Cl−5.0, 0.05)), and with accuracies that remained very good with the 2 methods of calculating tubular excretion of the tracer (table 5).

TABLE 5
Assessing the performance of CT-measured GFR relative to iohexol clearance,
in patients with chronic kidney disease vs healthy individuals.

| | | | | | Cortex- | |
|---|---|---|---|---|---|---|
| | | | | Cortex- | adjusted | |
| | | CT-measured | | adjusted | CT-measured | |
| | CT-measured | GFR in living | | CT-measured | GFR in living | |
| | GFR in CKD | kidney donor | P | GFR in CKD | kidney donor | P |
| Parameters | patients | candidates | value | patients | candidates | value |
| Mean relative | 11.6 | 2.4 | | 2.9 | −2.2 | |
| bias (%) | (5.5, 17.7) | (−0.05, 5.3) | | (−1.9, 7.7) | (−5.0, 0.05) | |
| Precision (%)* | 22.1 | 16.6 | 0.098 | 20.4 | 16.2 | 0.7 |
| | (0.0 to 22.0) | (−6.2 to 10.4) | | (−6.7 to 13.7) | (−10.1 to 6.1) | |
| Accuracy | 89.3 | 98.7 | 0.06 | 100 | 100 | 1 |
| within 30% | (77.8, 100) | (96.1, 100) | | [28/28] | [75/75] | |
| | [25/28] | [74/75] | | | | |
| Accuracy | 67.9 | 85.3 | 0.06 | 92.9 | 89.3 | 0.6 |
| within 20% | (50.6, 85.2) | (77.3, 93.3) | | (83.3, 100) | (82.4, 96.3) | |
| | [19/28] | [64/75] | | [25/28] | [67/75] | |
| Accuracy | 35.7 | 57.3 | 0.08 | 50.0 | 54.7 | 0.7 |
| within 10% | (18.0, 53.5) | (46.1, 68.5) | | (31.5, 68.5) | (43.4, 65.9] | |
| | [10/28] | [43/75] | | [14/28] | [41/75] | |

Note:
Unless otherwise specified, data in parentheses are 95% Cis, and data in brackets are numbers of patients.
P values were calculated for the precision comparison using the F test for comparison of variances, and accuracy comparisons using the Fisher exact test.
GFR = glomerular filtration rate
*Precision is the distance between quartile 1 and quartile 3 of the bias, with the values of quartiles 1 and 3 given in parentheses.

### Interobserver Reproducibility for Cortex-Adjusted CT-Measured GFR in CKD Patients

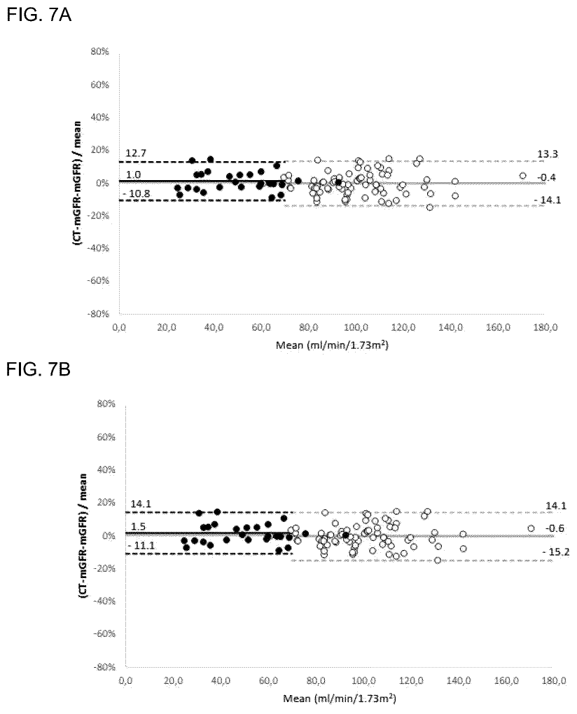

Inter-observer agreement was assessed by the Lin concordance correlation coefficient at 0.99 (95% Cl: 0.97, 0.99) whether CT-measured GFR was determined without or with consideration of cortical enhancement attributable to its vascular compartment. In healthy individuals, the values were 0.92 (95% Cl: 0.88, 0.95), and 0.91 (95% Cl: 0.87, 0.94) for CT-measured GFR determined without or with cortex adjustment, respectively (values slightly lower due to the higher range of GFR). The Bland Altman representation showed that there was no bias between the 2 assessors for CKD patients as for healthy individuals, and that the lower and upper limits of the agreement interval were very close in the two groups (FIGS. 7A-7B)

### Discussion

There is an unmet need for the development of rapid and reproducible GFR measurement methods that could be used on a large scale in clinical practice. We have developed a new method to accurately measure GFR, in the examples from 4-phases polychromatic CT urography, performed as part of routine care in healthy individuals. The main objective of our new study was to evaluate the performance of this CT urography-based method of measuring GFR in CKD patients. CT-mGFR was unbiased (mean bias: 2.9% (95% Cl: −1.9, 7.7)) and accurate compared to iohexol clearance (reference method) (accuracy within 30%, 20% and 10%: 100%, 92.9% (95% Cl 83.3, 100) and 50% (31.5, 68.5), respectively), with the condition that the part of kidney cortical enhancement in the excretory phase attributable to the contrast agent in the vascular compartment was subtracted from the calculation of CT-mGFR. In healthy individuals with normal GFR the CT-mGFR was unbiased, and highly accurate whether the cortical vascular compartment was included in or subtracted from the CT-mGFR calculation. The interobserver agreement of CT-mGFR obtained with different segmentation software was excellent with a Lin concordance coefficient of 0.99 (95% Cl: 0.97, 0.99) in CKD patients.

Historical methods for measuring GFR from CT scan used pharmacokinetic models such as Patlak's ([7]), requiring specific dynamic acquisitions and leading to imperfectly accurate results [4,5]. More recently, approaches have been developed to measure the clearance of iodinated contrast media by dividing the filtration amount of the contrast media by its plasma concentration, but these still had limitations such as the need to use specific CT acquisition procedures, whether dynamic or not, the need to perform a hematocrit blood test at the time of CT, and results whose accuracy was not always optimal (Yuan X et al. ([2]), You S et al. ([3]). Moreover, these studies included very few CKD patients: Only 2 patients in the study of Yuan et al had a plasma clearance of technetium 99m diethylenetriaminepenta-acetic acid below 60 ml/min (Yuan X et al. ([2])). In the You, Ma and Zhang study, where CT-mGFR was split into single-kidney GFR, only 5 of the 36 patients had one single-kidney GFR lower than 30 ml/min (You S et al. ([3])). Although the number of patients in our study was limited, these are the first promising results for future use of CT-urography in a value-added opportunistic imaging approach to measure GFR in CKD patients.

Our results show that our method for measuring GFR by CT-scan can be used with different iodinated contrast agents, such as iomeprol, iobitridol and iodixanol. Moreover, pharmacokinetic studies indicate that these molecules, with molecular weights of 835 g/mol and 1550 g/mol, are eliminated almost exclusively by the urinary excretion, and more specifically by glomerular filtration (Svaland M G et al. ([25]), Spencer C M, Goa K L ([26])). Similarly, since 6 of the 28 patients had had monochromatic CT scans, this shows that our CT-scan-based GFR measurement method can be used with spectral CT scans.

In conclusion, our method for measuring GFR from CT-urography performed as part of routine care would be valid in CKD patients, provided that the kidney cortical enhancement at the excretory phase, attributable to the presence of the tracer in the vascular compartment of the cortex, would be subtracted from the calculation of iodinated contrast agent clearance. Implementation in routine clinical practice would be relevant mainly in the field of urologic oncology (e.g. prediction of post-nephrectomy GFR, decision whether or not to administer cisplatin, carboplatin dose calculation based on Calvert formula using mGFR rather than eGFR).

### REFERENCE LIST

1 Ebert N, Bevc S, Bökenkamp A, et al. Assessment of kidney function: clinical indications for measured GFR. Clinical Kidney Journal. 2021; 14 (8): 1861-1870. doi: 10.1093/ckj/sfab042.
2. Yuan X, Zhang J, Tang K, et al. Determination of Glomerular Filtration Rate with CT Measurement of Renal Clearance of Iodinated Contrast Material versus 99m Tc-DTPA Dynamic Imaging “Gates” Method: A Validation Study in Asymmetrical Renal Disease. Radiology. 2017; 282(2):552-560. doi: 10.1148/radiol.2016160425.
3. You S, Ma X, Zhang C, et al. Determination of single-kidney glomerular filtration rate (GFR) with CT urography versus renal dynamic imaging Gates method. Eur Radiol. 2018; 28(3):1077-1084. doi: 10.1007/s00330-017-5061-z.
4. Hackstein N, Puille M F, Bak B H, Scharwat O, Rau W S. Measurement of single kidney contrast media clearance by multiphasic spiral computed tomography: preliminary results. Eur J Radiol. 2001; 39(3):201-208. doi: 10.1016/s0720-048x(01)00323-0.
5. Hackstein N, Wiegand C, Rau W S, Langheinrich A C. Glomerular filtration rate measured by using triphasic helical CT with a two-point Patlak plot technique. Radiology. 2004; 230(1):221-226. doi: 10.1148/radiol.2301021266.
6. Jeong S, Park S B, Chang I H, et al. Estimation of renal function using kidney dynamic contrast material-enhanced CT perfusion: accuracy and feasibility. Abdom Radiol. 2021; 46(5):2045-2051. doi: 10.1007/s00261-020-02826-7.
7. Patlak C S, Blasberg R G, Fenstermacher J D. Graphical evaluation of blood-to-brain transfer constants from multiple-time uptake data. J Cereb Blood Flow Metab. 1983; 3(1):1-7. doi: 10.1038/jcbfm. 1983.1.
8. Aydin F, Güngör F, Cengiz A K, et al. Comparison of glomerular filtration rate measurements with the two plasma sample and single plasma sample, gamma camera Gates, creatinine clearance, and prediction equation methods in potential kidney donors with normal renal function. Nucl Med Commun. 2008; 29(2):157-165. doi: 10.1097/MNM.0b013e3282f1bbde.
9. Itoh K. Comparison of methods for determination of glomerular filtration rate: Tc-99m-DTPA renography, predicted creatinine clearance method and plasma sample method. Ann Nucl Med. 2003; 17(7):561-565. doi: 10.1007/BF03006669.
10. Kumar M, Arora G, Damle N, et al. Comparison between two-sample method with 99m Tc-diethylenetriaminepentaacetic acid, Gates' method and estimated glomerular filtration rate values by formula based methods in healthy kidney donor population. Indian J Nucl Med. 2017; 32(3):188. doi: 10.4103/ijnm.IJNM_17_17.
11. Lentine K L, Kasiske B L, Levey A S, et al. KDIGO Clinical Practice Guideline on the Evaluation and Care of Living Kidney Donors. Transplantation. 2017; 101(8S):S7-S105. doi: 10.1097/TP.0000000000001769.
12. Cavalier E, Rozet E, Dubois N, et al. Performance of iohexol determination in serum and urine by HPLC: validation, risk and uncertainty assessment. Clin Chim Acta. 2008; 396(1-2):80-85. doi: 10.1016/j.cca.2008.07.011.
13. Seegmiller J C, Burns B E, Schinstock C A, Lieske J C, Larson T S. Discordance Between Iothalamate and Iohexol Urinary Clearances. Am J Kidney Dis. 2016; 67(1):49-55. doi: 10.1053/j.ajkd.2015.08.020.
14. Stehlé T, El Karoui K, Sakka M, et al. Creatinine clearance after cimetidine administration in a new short procedure: comparison with plasma and renal clearances of iohexol. Clinical Kidney Journal. 2020; 13(4):587-596. doi: 10.1093/ckj/sfz087.
15. Bröchner-Mortensen J. A Simple Method for the Determination of Glomerular Filtration Rate. Scandinavian Journal of Clinical and Laboratory Investigation. 1972; 30(3):271-274. doi: 10.3109/00365517209084290.
16. Black D F, Rad A E, Gray L A, Campeau N G, Kallmes D F. Cerebral venous sinus density on noncontrast CT correlates with hematocrit. AJNR Am J Neuroradiol. 2011; 32(7):1354-1357. doi: 10.3174/ajnr.A2504.
17. Passing H, Bablok null. A new biometrical procedure for testing the equality of measurements from two different analytical methods. Application of linear regression procedures for method comparison studies in clinical chemistry, Part I. J Clin Chem Clin Biochem. 1983; 21(11):709-720. doi: 10.1515/cclm. 1983.21.11.709.
18. Bland J M, Altman D G. Statistical methods for assessing agreement between two methods of clinical measurement. Lancet. 1986; 1(8476):307-310.
19. Lin L I. A concordance correlation coefficient to evaluate reproducibility. Biometrics. 1989; 45(1):255-268.
20. Inker L A, Eneanya N D, Coresh J, et al. New Creatinine- and Cystatin C-Based Equations to Estimate GFR without Race. N Engl J Med. 2021; 385(19):1737-1749. doi: 10.1056/NEJMoa2102953.
21. Soveri I, Berg U B, Björk J, et al. Measuring GFR: A Systematic Review. American Journal of Kidney Diseases. 2014; 64(3):411-424. doi: 10.1053/j.ajkd.2014.04.010.
22. Lorusso V, Luzzani F, Bertani F, Tirone P, de Haën C. Pharmacokinetics and tissue distribution of iomeprol in animals. Eur J Radiol. 1994; 18 Suppl 1:S13-20. doi: 10.1016/0720-048x(94)90090-6.
23. Lorusso V, Taroni P, Alvino S, Spinazzi A. Pharmacokinetics and safety of iomeprol in healthy volunteers and in patients with renal impairment or end-stage renal disease requiring hemodialysis. Invest Radiol. 2001; 36(6):309-316. doi: 10.1097/00004424-200106000-00002.
24. Stehlé T, Wei F, Brabant S, et al. Glomerular Filtration Rate Measured Based on Iomeprol Clearance Assessed at CT Urography in Living Kidney Donor Candidates. Radiology. 2023; 309(3):e230567. doi: 10.1148/radiol.230567.
25. Svaland M G, Haider T, Langseth-Manrique K, Andrew E, Hals P A. Human pharmacokinetics of iodixanol. Invest Radiol. 1992; 27 (2):130-133. doi: 10.1097/00004424-199202000-00007.
26. Spencer C M, Goa K L. Iodixanol: A Review of its Pharmacodynamic and Pharmacokinetic Properties and Diagnostic Use as an x-Ray Contrast Medium. Drugs. 1996; 52(6):899-927. doi: 10.2165/00003495-199652060-00013.

1. Method for ex vivo measuring glomerular filtration rate (GFR) of a subject to whom a contrast agent has been administered, and to whom:
computed tomography (CT) or magnetic resonance imaging (MRI) including an excretory phase, an unenhanced phase, an arterial phase, and a nephrographic or a venous portal phase has been performed, said method comprising the step of calculating the clearance of the contrast agent from CT or MRI data using manual, automated or semi-automated segmentation software, thereby obtaining a measured GFR.

2. Method according to claim 1, wherein the contrast agent is an iodinated or non-iodinated ideal GFR marker.

3. Method according to claim 1, wherein contrast agent clearance is calculated by dividing urinary excretion rate of contrast agent between arterial and excretory phases by calculated serum concentration of the contrast agent at mid period.

4. Method according to claim 3, wherein the urinary excretion rate of contrast agent between arterial and excretory phases is the sum of urinary excretion of the contrast agent into the bladder, urinary excretion of the contrast agent into the upper excretory tract, and urinary excretion of the contrast agent into the kidney tubules.

5. Method according to claim 4, wherein the urinary excretion of the contrast agent into the kidney tubules is the volume of both kidneys multiplied by mean attenuation of kidneys at the excretory phase minus mean attenuation of kidneys at the unenhanced phase, then divided by time period.

6. Method according to claim 4, wherein the urinary excretion of the contrast agent into the kidney tubules is the sum of urinary excretion of the contrast agent into the kidney medulla and urinary excretion of the contrast agent into the kidney cortex.

7. Method according to claim 6, wherein:
the urinary excretion of the contrast agent into the kidney medulla is the volume of kidney medullary multiplied by mean CT-attenuation of kidneys at the excretory phase minus mean attenuation of kidneys at the unenhanced phase, then divided by time period, and
the urinary excretion of the contrast agent into the kidney cortex is the volume of kidney cortex multiplied by mean CT-attenuation of kidney at the excretory phase minus CT-attenuation of the kidneys in the unenhanced phase, and minus cortical CT attenuation from the vascular compartment in the excretory phase, then divided by time between arterial and excretory phases.

8. Method according to claim 3, wherein specific areas of CT scan or MRI scan are segmented at different times in order to determine urinary excretion rate of the contrast agent.

9. Method according to claim 3, wherein the mid period serum contrast agent concentration is deduced from a linear or polynomial regression after transformation of each intermediate value of serum concentration equivalents into a natural logarithm.

10. Method according to claim 1, wherein the step of calculating is performed automatically using software developed from algorithms developed by artificial intelligence or other methods.

11. Method according to claim 1, wherein the excretory phase includes kidney parenchyma, upper kidney excretory system, and bladder, the unenhanced phase includes the kidney parenchyma and, optionally the bladder, the arterial phase includes either a single slice of the aorta or the full height of the kidneys, and nephrographic or portal venous phases include at least one aortic single slice, together with the bladder if not included in the unenhanced phase.

12. Method according to claim 1, wherein the concentration of the contrast agent is determined in CT by a method other than the Hounsfield unit count, for example direct quantification of the contrast agent by spectral imaging on a CT with or without photon counting or spectral CT.

13. Method according to claim 1, in which the measurement of urinary excretion of the contrast agent in the bladder is determined on the basis of the part of the bladder that is enhanced by the contrast agent or of the entire volume of the bladder.

14. Method according to claim 1, in which a volume of aorta or a single slice is used to measure CT density or MRI signal of the aorta.

15. Method according to claim 1, wherein the contrast agent is iodinated contrast agent, for example chosen among iomeprol, iobitridol and iodixanol.
